(19) United States
(12) Patent Application Publication
(10) Pub. No.: US 2018/0070927 A9
(43) Pub. Date: Mar. 15, 2018

(54) FLEXIBLE TUBE ASSEMBLY

(71) Applicant: TERUMO KABUSHIKI KAISHA, Tokyo (JP)

(72) Inventors: TOMONORI HATTA, KANAGAWA (JP); YUICHI TADA, KANAGAWA (JP); AYAKA AKUTAGAWA, KANAGAWA (JP); KAORU MIYAZAKI, KANAGAWA (JP)

(21) Appl. No.: 14/671,685

(22) Filed: Mar. 27, 2015

Related U.S. Application Data
(63) Continuation of application No. PCT/JP2012/078239, filed on Oct. 31, 2012.

(51) Int. Cl. A61B 10/00; A61B 17/00

(52) U.S. Cl. CPC A61B 10/0051; A61B 2010/0208; A61B 17/00234

(57) ABSTRACT

A flexible tube assembly according to the present disclosure includes a multiple tube having at least a first tubular member that has an elongated shape and is hollow and a second tubular member that has an elongated shape and is disposed inside the first tubular member in such a manner as to be capable of advancing and retreating relative to the first tubular member; and a hand operation unit that is provided on a proximal side of the multiple tube and operates at least the first tubular member and the second tubular member. The hand operation unit includes a curving operation unit that is connected to the first tubular member or the second tubular member and curves the first tubular member or the second tubular member that is connected, a slide unit that causes the second tubular member to make slide movement relative to the first tubular member, and a switching operation unit that makes switching between a state in which the first tubular member and the second tubular member are fixed to each other and a state in which fixing between the first tubular member and the second tubular member is released.

### CROSS-REFERENCE TO RELATED APPLICATIONS

This application is a continuation of International Application No. PCT/JP2012/078239, with an international filing date of Oct. 31, 2012. The entire contents of each and every foregoing applications are incorporated herein by reference.

### BACKGROUND

The present disclosure relates to a flexible tube assembly used in e.g. a treatment for a disease of the respiratory system.

Biopsy techniques, in which biological tissues are harvested from a lesion site of a patient and tested in order to make a diagnosis of the disease condition of the patient and so forth, are widely known. In the case of harvesting biological tissues, first an endoscope like one described in Japanese Patent Laid-Open No. Sho 63-238839 is made to enter the inside of a living body. Subsequently, a biopsy instrument having a diameter set small, such as biopsy forceps or a biopsy needle, is made to enter the inside of the living body. Then, the biopsy instrument is stuck into a target site and biological tissues are excised to harvest the biological tissues.

### SUMMARY

The biopsy technique is used for a definitive diagnosis of a disease such as a cancer and a medical instrument such as an endoscope penetrates to a lesion site for which a biopsy is to be performed. A flexible tube such as a catheter used for the endoscope or the like generally has substantially the same diameter from the proximal side to the distal side. However, the size of lumens in the living body into which the medical instrument such as an endoscope is introduced is not uniform in the longitudinal direction. For example, as is well known, the lung has a structure in which, after branching from the trachea into the left and right main bronchi, the sectional area decreases with branching from the main bronchi into organs such as the superior lobe bronchus, the middle lobe bronchus, and the inferior lobe bronchus.

The flexible tube part of the endoscope in Japanese Patent Laid-Open No. Sho 63-238839 is a single tube. When being used for a lung biopsy, the endoscope having the flexible tube with a desired size is selected according to the size of a lung site into which it is to be inserted. Therefore, in the case of introducing the endoscope into a peripheral site of a lung, the endoscope having the flexible tube with a comparatively-small diameter is selected. With the endoscope having the small-diameter flexible tube, the gap between the flexible tube and the tracheal inner wall is comparatively large at a central site of the lung. Therefore, there is a problem that it is difficult to direct the endoscope having the flexible tube toward the peripheral site and accordingly it is difficult to make the endoscope rapidly advance to the target site.

As a countermeasure against this problem, it would be effective to give a multiple structure to the flexible tube used for the endoscope in conformity with the sizes of the respective parts in the body into which it is to be introduced. However, there has not yet been a product that is used for an endoscope or the like and is obtained by combining, into a multiple structure, flexible tubes conforming to the size of the conduit whose sectional area changes like the lung.

Therefore, the present disclosure is invented in order to solve the above-described problem and an object thereof is to provide a flexible tube assembly that can be made to rapidly penetrate to a desired site in a conduit whose sectional area changes like the lung.

A flexible tube assembly according to the present disclosure to achieve the above-described object includes a multiple tube having at least a first tubular member that has an elongated shape and is hollow and a second tubular member that has an elongated shape and is disposed inside the first tubular member in such a manner as to be capable of advancing and retreating relative to the first tubular member, and a hand operation unit that is provided on a proximal side of the multiple tube and operates at least the first tubular member and the second tubular member. In the present disclosure, the hand operation unit includes a curving operation unit that is connected to the first tubular member or the second tubular member and curves the first tubular member or the second tubular member that is connected, a slide unit that causes the second tubular member to make slide movement relative to the first tubular member, and a switching operation unit that makes switching between a state in which the first tubular member and the second tubular member are fixed to each other and a state in which fixing between the first tubular member and the second tubular member is released.

The flexible tube assembly according to the present disclosure allows switching by the switching operation unit between the state in which the first tubular member and the second tubular member configuring the multiple tube are fixed to each other and the state in which the fixing is released. Therefore, in the case of making the flexible tube assembly according to the present disclosure enter the inside of a lung, at the central part of the lung, where the sectional area is comparatively large, the flexible tube assembly can be made to penetrate with the first tubular member having a comparatively-large diameter fixed to the second tubular member. Thus, the flexible tube assembly can be rapidly directed toward a peripheral site and moved forward thereto with the distance from the inner wall surface of the lung prevented from becoming large. Furthermore, at the peripheral site, the fixing between the first tubular member and the second tubular member is released by the switching operation unit and the second tubular member is protruded from the distal end of the first tubular member by the slide unit, so that the second tubular member having a comparatively-small diameter can be rapidly moved forward toward the further peripheral side.

### BRIEF DESCRIPTION OF THE DRAWINGS

FIG. 1 is a perspective view showing a flexible tube assembly according to a first embodiment of the present disclosure;

FIG. 2 is a sectional view along line 2-2 in FIG. 1;

FIG. 3 is a sectional view along line 3-3 in FIG. 1;

FIG. 4 is an enlarged sectional view showing a switching operation unit in FIG. 2;

FIG. 5 is a sectional view showing a second tubular member of the flexible tube assembly;

FIG. 6 is a perspective view showing a biopsy instrument as one example of a medical instrument attached to the distal end of the second tubular member of the flexible tube assembly;

FIG. 7 is a perspective view showing a flexible tube assembly according to a second embodiment of the present disclosure;

FIG. 8 is a sectional view along line 8-8 in FIG. 7;

FIG. 9 is a sectional view along line 9-9 in FIG. 7;

FIG. 10 is a perspective view showing a modification example of the flexible tube assembly according to the first embodiment of the present disclosure;

FIG. 11 is an enlarged sectional view along line 11-11 in FIG. 10; and

FIG. 12 is a detail view showing another modification example of the flexible tube assembly and showing a switching operation unit in FIG. 11 in an enlarged manner.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### DETAILED DESCRIPTION OF THE PREFERRED EMBODIMENTS

Embodiments of the present disclosure will be described below with reference to the accompanying drawings. Note that the following description shall not limit technical scopes and the meanings of terms set forth in the scope of claims. Furthermore, the dimensional ratio of the drawings is exaggerated for convenience of explanation and is different from the actual ratio in some cases.

### First Embodiment

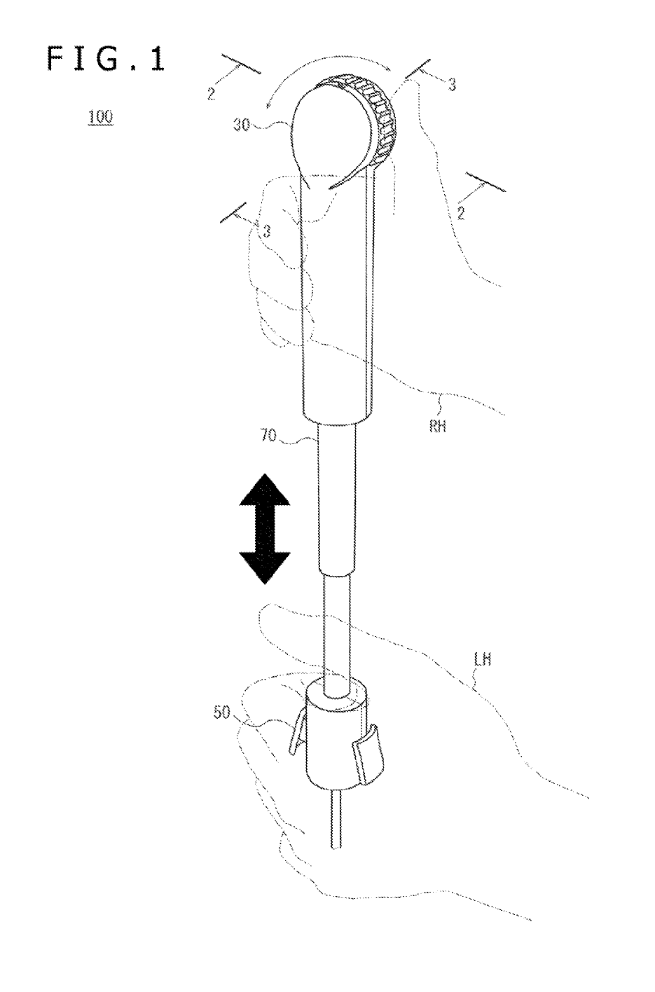

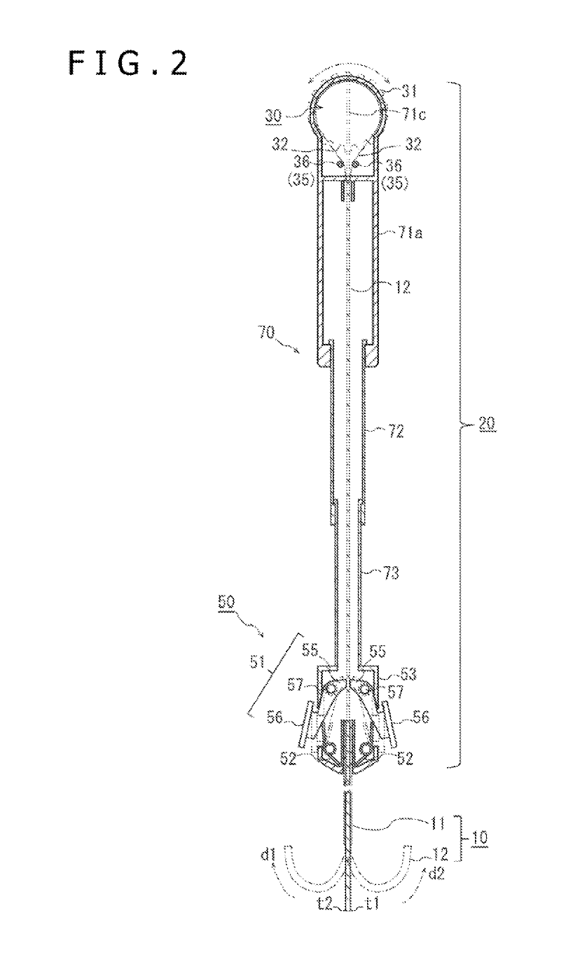

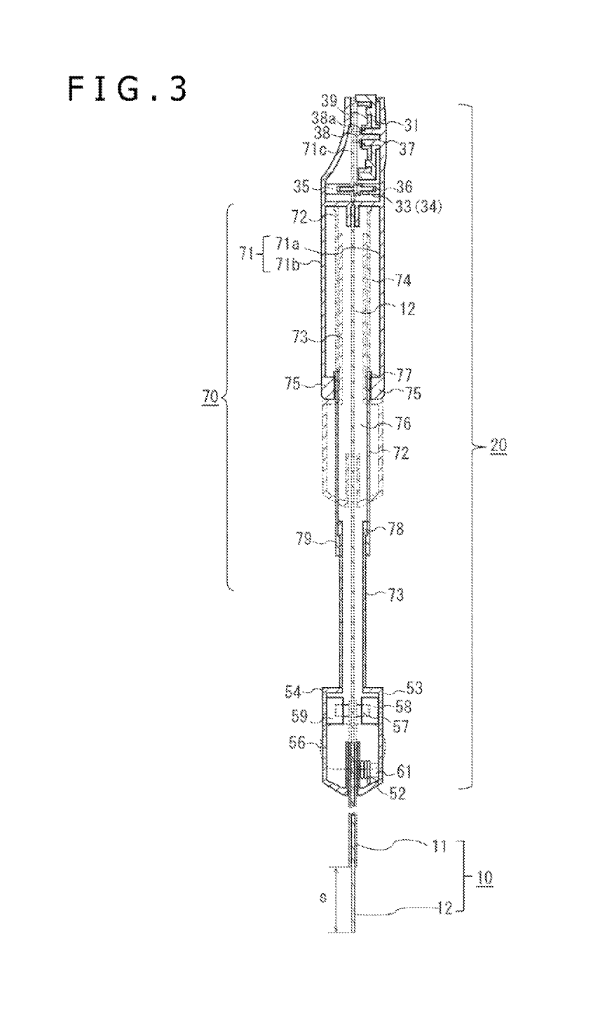

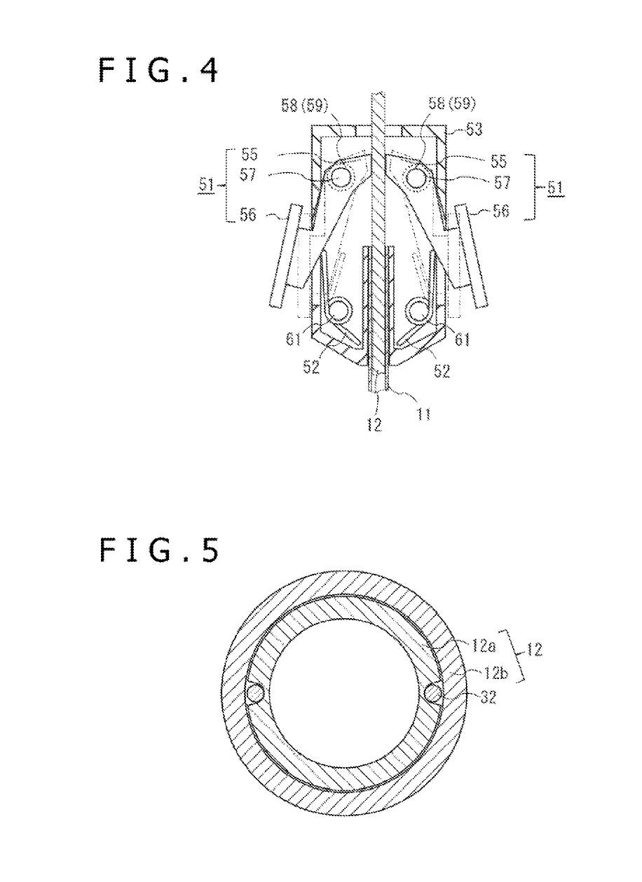

FIG. 1 is a perspective view showing a flexible tube assembly according to a first embodiment of the present disclosure. FIG. 2 is a sectional view along line 2-2 in FIG. 1. FIG. 3 is a sectional view along line 3-3 in FIG. 1. FIG. 4 is an enlarged sectional view showing a switching operation unit in FIG. 2. FIG. 5 is a sectional view showing a second tubular member of the flexible tube assembly.

Into the inside of the distal part of a flexible tube assembly 100 according to the first embodiment, an imaging element such as a camera and biopsy forceps, a biopsy needle, or the like to harvest cells of a lesion site are inserted. The flexible tube assembly 100 is used in e.g. a procedure to harvest cells of a lesion site.

An outline will be described with reference to FIGS. 1 to 5. The flexible tube assembly 100 according to the present embodiment has a multiple tube 10 and a hand operation unit 20. The multiple tube 10 has an outer tube 11 (equivalent to the first tubular member) that has an elongated shape and is hollow and an inner tube 12 (equivalent to the second tubular member) disposed inside the outer tube 11 in such a manner as to be capable of advancing and retreating relative to the outer tube 11. The hand operation unit 20 is provided on the proximal side of the multiple tube 10 and operates at least the outer tube 11 and the inner tube 12. Note that the proximal side in the flexible tube assembly 100 here refers to the side on which the hand operation unit 20 for allowing an operator to operate the flexible tube assembly 100 and so forth are located in the longitudinal direction of the flexible tube assembly 100, which has the elongated shape. Furthermore, the distal side refers to the side to which medical instruments such as an endoscope and a puncture tool are attached and that is introduced into a living body to perform a diagnosis and a treatment in the longitudinal direction of the flexible tube assembly 100.

The hand operation unit 20 has a curving operation unit 30 that is connected to the inner tube 12 and curves the connected inner tube 12, a slide unit 70 that causes the inner tube 12 to make a slide movement relative to the outer tube 11, and a switching operation unit 50 that makes switching between the state in which the outer tube 11 and the inner tube 12 are fixed to each other and the state in which the fixing between the outer tube 11 and the inner tube 12 is released. Details thereof will be described below.

The outer tube 11 is a hollow tubular member and houses the inner tube 12 inside it. In the present embodiment, the distal part of the outer tube 11 curves along with a curving action of the distal part of the inner tube 12 when the distal part of the outer tube 11 corresponds with that of the inner tube 12 due to the switching operation unit 50 or the amount of protrusion of the inner tube 12 from the outer tube 11 is small. Therefore, it is preferable that particularly the distal part in the outer tube 11 has such a shape or structure as to curve more readily than the inner tube 12. To take an example, it is preferable for the outer tube 11 to have a bellows structure. This is because, with a bellows structure, the outer tube 11 can easily curve at an arbitrary position in the longitudinal direction and a kink occurs less readily than with a straight tubular shape that is not a bellows.

The outer tube 11 is connected to cases 53 and 54 forming the switching operation unit 50 by an adhesive or the like. However, the connecting method is not limited thereto and may be e.g. thermal fusion bonding.

The inner tube 12 has a first inner tube 12a having a hollow tubular shape and a second inner tube 12b having a hollow tubular shape. In the present embodiment, the first inner tube 12a is fitted into the second inner tube 12b and the first inner tube 12a and the second inner tube 12b are so configured as to operate integrally with each other. In the first inner tube 12a, grooves are formed at part of the outer periphery in the circumference at intervals of e.g. 180 degrees as shown in FIG. 5. A wire 32 inserted in the curving operation unit 30 to be described later is disposed in these grooves and is bonded by an adhesive or the like. The first inner tube 12a and the second inner tube 12b are soft members formed of polyurethane, polyolefin, polyester, polycarbonate, polysulfone, silicone, or the like. The inner tube 12 is a component to be made to penetrate to a peripheral site such as the superior lobe bronchus, the middle lobe bronchus, and the inferior lobe bronchus of a lung. Therefore, it is preferable for the inner tube 12 to have as small a diameter as possible. As an example, the diameter is about φ3 mm.

To the inside surface of the proximal side of the first inner tube 12a, a hollow insertion tube 71c for transferring medical instruments such as an endoscope and a biopsy needle to the distal end of the flexible tube assembly 100 while the inner tube 12 is curved by the wire 32 is connected. The insertion tube 71c is fixed by an adhesive, thermal fusion bonding, or the like at the proximal part of a slide member piece 71b forming a slide member 71 to be described later in such a manner that curving of the inner tube 12 is allowed. In FIGS. 2 and 3, for convenience of diagrammatic representation, the first inner tube 12a and the second inner tube 12b are shown as a solid monolithic member and the insertion tube 71c is shown as a solid member. Furthermore, in FIG. 4, the first inner tube 12a and the second inner tube 12b are shown as a solid monolithic member.

The first inner tube 12a and the second inner tube 12b are one example of the structure to curve the inner tube 12 and the structure is not limited thereto. For example, besides the above-described configuration, a configuration may be employed in which simply a wire is bonded to a tube obtained by making grooves at the outer periphery of the first inner tube 12a without providing the second inner tube 12b.

The lengths of the outer tube 11 and the inner tube 12 are adjusted as appropriate depending on the site into which the flexible tube is to be inserted and the patient. As an example, the lengths of the outer tube 11 and the inner tube 12 are 600 mm and 800 mm, respectively.

The curving operation unit 30 has a rotating dial 31 rotatably disposed in order to curve the inner tube 12 connected to the curving operation unit 30 and the wire 32 that causes the inner tube 12 to act in accordance with the motion of the rotating dial 31 in order to curve the inner tube 12. The curving operation unit 30 further has pulleys 33 and 34 for restricting the wiring route of the wire 32 and a holding member 39 for holding the rotating dial 31 rotatably onto a slide member piece 71a to be described later.

Recesses and projections are made in the outer circumference of the rotating dial 31 so that a finger to perform curving operation may easily get caught on it. However, the shape is not limited to recesses and projections. The rotating dial 31 is formed of a material having comparatively-high hardness, such as polypropylene. The rotating dial 31 is rotatably attached to the slide member piece 71a forming the slide member 71 through the following process. Specifically, a bolt-attached part of the slide member piece 71a is inserted into a through-hole of the rotating dial 31. Furthermore, the bolt-attached part of the slide member piece 71a is inserted into a through-hole of the holding member 39. In this state, a washer 38a is attached and then a bolt 38 is screwed to a screw hole 37 made in the slide member piece 71a.

Part of the wire 32 is wound around the rotating dial 31 and is bonded thereto by an adhesive or the like, and both ends of the wire 32 extend to the distal end of the inner tube 12.

The curving operation by the rotating dial 31 and the wire 32 will be described below. As described above, the wire 32 is clamped by the first inner tube 12a and the second inner tube 12b of the inner tube 12 and is bonded to the groove part at the distal part of the first inner tube 12a. This allows operation by the rotating dial 31 to be transmitted to the inner tube 12 via the wire 32. When the positions of both ends of the wire 32 are the same in the longitudinal direction, the inner tube 12 is in a state of extending in a straight line manner in the plane of FIG. 2 and being not curved.

When the rotating dial 31 is rotated clockwise, a distal end t2 of the wire 32 on the left lower side in FIG. 2 is pulled upward. Therefore, the inner tube 12 connected to the wire 32 curves in a direction d1 indicated by a two-dot chain line in the plane of FIG. 2.

On the other hand, when the rotating dial 31 is rotated anticlockwise, a distal end t1 of the wire 32 on the right lower side in FIG. 2 is pulled toward the upper side in FIG. 2. Therefore, the inner tube 12 connected to the wire 32 curves in a direction d2 indicated by a one-dot chain line in the plane of FIG. 2.

In this manner, the positional relationship between both distal parts of the wire 32 changes by the rotational operation of the rotating dial 31, so that the curving operation of the curving operation unit 30 is realized.

As described above, the curving operation unit 30 curves the inner tube 12 in the direction d1 or the direction d2 in the plane of FIG. 2 based on the direction in which the rotating dial 31 is rotated. Therefore, in the case of curving the distal side of the flexible tube assembly 100 in a desire direction in a living body, in order to curve the outer tube 11 and the inner tube 12 or only the inner tube 12 in the desired direction, the whole of the flexible tube assembly 100 is rotated in the circumferential direction while the rotating dial 31 is rotated to curve the outer tube 11 and the inner tube 12 or only the inner tube 12.

The pulleys 33 and 34 are rotatably attached to pulley support parts 35 and 36 provided inside the slide member pieces 71a and 71b. The pulleys 33 and 34 modify the extension direction of the wire 32 extending from the rotating dial 31 to the direction along the axis line of the inner tube 12.

The switching operation unit 50 has grip members 51, biasing members 52, and the cases 53 and 54. In the following, the respective constituent elements of the switching operation unit 50 will be described and fixing between the outer tube 11 and the inner tube 12 and release of the fixing by the switching operation unit 50 will be described.

The grip members 51 are disposed as a pair of members astride the outer tube 11 and the inner tube 12 in the cases 53 and 54. Each grip member 51 has a fixing part 55 that fixes the outer tube 11 to the inner tube 12 by pressing the inner tube 12 radially inward to grip it and a release lever 56 that displaces the fixing part 55 radially outward against a biasing force by the biasing member 52 to release the fixing between the outer tube 11 and the inner tube 12. Furthermore, each grip member 51 has a rotation pin 57 for being rotatably attached to the cases 53 and 54.

The outer tube 11 is bonded to the cases 53 and 54 and the rotation pins 57 of the grip members 51 are attached to pin support parts 58 of the case 53 and pin support parts 59 of the case 54. This connects the fixing parts 55 to the outer tube 11 with the intermediary of the cases 53 and 54. The fixing parts 55 press the inner tube 12 from the radially outside and grip it in order to fix the outer tube 11 to the inner tube 12. The contact parts with the inner tube 12 for gripping the inner tube 12 may have a flat surface shape. However, they may have a curved surface shape in conformity with the side surface shape of the inner tube 12.

The biasing members 52 are disposed as a pair of members astride the outer tube 11 and the inner tube 12 in the cases 53 and 54 similarly to the grip members 51. The biasing members 52 are connected to the grip members 51 and give, at the connected parts, a force to push out the release levers 56 radially outward. In the state in which the release levers 56 are biased radially outward by the biasing members 52, the fixing parts 55 are biased radially inward due to the rotation of the grip members 51 about the rotation pins 57 as pivot points.

Due to this, the fixing parts 55 press and grip the inner tube 12 to generate contact resistance attributed to friction at the contact parts with the inner tube 12. By this contact resistance, the fixing parts 55 are held at arbitrary positions in the longitudinal direction on the inner tube 12.

Due to the holding of the fixing parts 55 at arbitrary positions in the longitudinal direction, the grip members 51 having the fixing parts 55 limit the movement of the cases 53 and 54 connected by the rotation pins 57 in the longitudinal direction and hold the cases 53 and 54 at arbitrary positions in the longitudinal direction on the inner tube 12.

As described above, the outer tube 11 is connected to the cases 53 and 54 by an adhesive or the like. Therefore, through the holding of the cases 53 and 54 at arbitrary positions in the longitudinal direction on the inner tube 12, the outer tube 11 connected to the cases 53 and 54 is held at an arbitrary position in the longitudinal direction on the inner tube 12.

In this manner, the biasing members 52 displace the release levers 56 of the grip members 51 radially outward to thereby generate contact resistance at the contact parts between the fixing parts 55 and the inner tube 12. This causes the outer tube 11 and the inner tube 12 to be integrally fixed to each other. The biasing members 52 are e.g. torsion coil springs. However, they may be formed by other members as long as the outer tube 11 can be fixed to the inner tube 12.

The release levers 56 are parts pressed and gripped by either hand among the parts in the grip members 51. When the release levers 56 are pushed radially inward by either hand (see two-dot chain lines in FIGS. 2 and 4), the fixing parts 55 are displaced radially outward due to the rotation of the grip members 51 about the rotation pins 57 as pivot points.

This makes the fixing parts 55 and the inner tube 12 become the non-contact state and eliminates the above-described contact resistance, which releases the holding of the grip members 51 having the fixing parts 55 in the longitudinal direction on the inner tube 12.

Due to the release of the holding of the grip members 51, the holding to the inner tube 12 is released also about the cases 53 and 54 connected by the rotation pins 57. Due to the release of the holding of the cases 53 and 54, the holding to the inner tube 12 is released also about the outer tube 11 connected to the cases 53 and 54.

As above, by only grip operation of the release levers 56, the fixing between the outer tube 11 and the inner tube 12 and the release of the fixing can be carried out without changing the way of holding the flexible tube assembly with hands.

The cases 53 and 54 are members obtained by dividing, into two sections, a cylindrical shape serving as the chassis of the switching operation unit 50 and internally house the fixing parts 55, the rotation pins 57, and the biasing members 52 of the grip members 51. Furthermore, notch parts are made in the side surfaces of the cases 53 and 54 so that the release levers 56 of the grip members 51 may be disposed outside the cases 53 and 54. The cases 53 and 54 have a cylindrical part whose section is a true circle so as to be easily gripped with either hand. However, the shape is not limited thereto and they may be e.g. a columnar body whose section is an ellipse or a columnar body whose section is a polygon. Furthermore, the cases 53 and 54 are connected to the outer tube 11 and allow the inner tube 12 to protrude from the outer tube 11. In addition, the cases 53 and 54 are connected to the distal part of a slide member 73 to be described later by thermal fusion bonding or the like. However, the configuration is not limited to the above one and the slide member 73 and the cases 53 and 54 may be a monolithic member.

Moreover, the cases 53 and 54 have the pin support parts 58 and 59 for rotatably attaching the pair of grip members 51 at two places about each pin support part and have two holding pins 61 for holding the pair of biasing members 52.

Next, the slide unit 70 will be described and slide movement operation of the inner tube 12 relative to the outer tube 11 will be described.

The slide unit 70 causes slide movement of the inner tube 12 relative to the outer tube 11. The slide unit 70 has the slide members 71, 72, and 73. The slide member 71 is disposed on the proximal side among the slide members and the rotating dial 31 is rotatably attached thereto. The slide member 71 internally includes the pulley support parts 35 and 36 and supports the pulleys 33 and 34 by the pulley support parts 35 and 36. Furthermore, the slide member 71 is formed of the slide member pieces 71a and 71b obtained by dividing a cylindrical member into two sections in the circumferential direction so that the pulleys 33 and 34 and the rotating dial 31 can be disposed inside the slide member 71. The proximal side of the slide member piece 71b is formed into a recess shape in conformity with the shape of the rotating dial 31. The slide member 71 has a cylindrical part whose section is a true circle similarly to the cases 53 and 54 of the switching operation unit 50. However, the shape is not limited thereto. The slide member 71 may be subjected to processing treatment such as increasing the surface roughness so as to be less slippery when being gripped.

The slide member 71 has an internal space 74 into which the slide member 72 and the slide member 73 can be slide-moved and housed in slide movement. Furthermore, the slide member 71 has, inside the distal side, a locking part 75 for locking a projection part 77 provided on the slide member 72.

The slide member 72 is disposed between the slide member 71 and the slide member 73. The slide member 72 is formed with a smaller diameter than the slide member 71 and the slide member 73 is formed with a smaller diameter than the slide member 72. The slide member 72 has an internal space 76 into which the slide member 73 can be slide-moved and housed when the slide member 72 moves to advance and retreat along the axis line of the inner tube 12. Furthermore, the slide member 72 has, on the proximal side, the projection part 77 protruding radially outward for being locked by the locking part 75 of the slide member 71 and has, inside the distal side, a locking part 79 for locking a projection part 78 of the slide member 73.

The slide member 73 is slidably connected to the slide member 72 on the proximal side and is connected to the cases 53 and 54 of the switching operation unit 50 on the distal side. The slide member 73 has the projection part 78 protruding radially outward on the proximal side for being locked by the locking part 79 of the slide member 72. The slide members 71 to 73 are so disposed as to be lined slidably in the long axis direction of the outer tube 11 and the inner tube 12.

As above, the slide unit 70 has a nesting structure composed of the slide members 71 to 73. Due to this, when the slide member 72 is located on the distal-most side in the internal space 74 of the slide member 71 and the slide member 73 is located on the distal-most side in the internal space 76 of the slide member 72, the cases 53 and 54 attached to the distal end of the slide member 73 are disposed at the position remotest from the slide member 71 (see the solid-line shape in FIG. 3). This provides the shortest distance as a distance s from the distal end of the outer tube 11 connected to the cases 53 and 54 to the distal end of the inner tube 12 connected to the slide member 71 with the intermediary of the rotating dial 31 and the wire 32.

Conversely, when the slide member 72 is located on the proximal-most side to which the slide member 72 can move in the internal space 74 of the slide member 71 and the slide member 73 is located on the proximal-most side to which the slide member 73 can move in the internal space 76 of the slide member 72, the cases 53 and 54 are disposed at the position closest to the slide member 71 (see the shape shown by two-dot chain lines in FIG. 3). This provides the longest distance as the distance s from the distal end of the outer tube 11 to the distal end of the inner tube 12.

As above, by the simple action of causing the cases 53 and 54 of the switching operation unit 50 to get closer to or remoter from the slide member 71, the amount of protrusion of the inner tube 12 from the outer tube 11 can be adjusted and slide movement operation of the inner tube 12 can be performed without giving excess attention to the operation at hand.

Furthermore, as shown in FIG. 3, the slide members 71 to 73 are so disposed as to be lined slidably in the extension direction of the axis line of the outer tube 11 and the inner tube 12. This allows the slide member 71 and the cases 53 and 54 of the switching operation unit 50 to get closer to or remoter from each other in the extension direction of the axis line of the outer tube 11 and the inner tube 12. Therefore, the slide movement operation can be smoothly performed without uselessly curving the inner tube 12 compared with the case of causing the slide member 71 and the cases 53 and 54 to get closer to or remoter from each other in a direction different from the extension direction of the outer tube 11 and the inner tube 12.

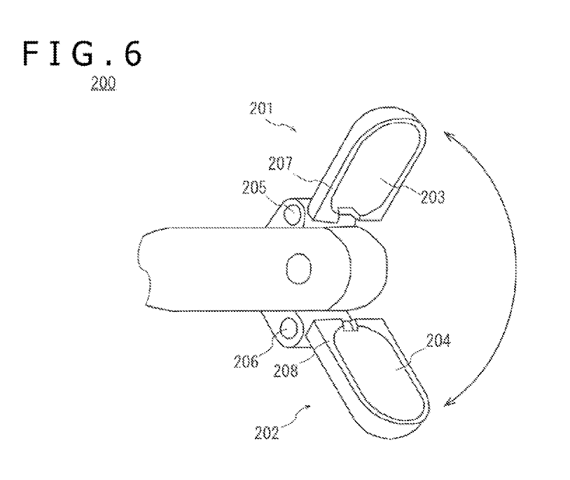

Next, as a usage example of the flexible tube assembly according to the first embodiment, harvesting of cells of a lesion site of a lung will be described. FIG. 6 is a perspective view showing the distal part of a biopsy instrument. To harvest cells of a lesion site, forceps 200 like ones shown in FIG. 6 are used as one example. The distal part of the forceps 200 is divided and distal portions 201 and 202 are formed. A harvesting part 203 to harvest a specimen is formed in the distal portion 201 and a harvesting part 204 is formed in the distal portion 202. The distal portion 201 rotates by a hinge point 205 and the distal portion 202 rotates by a hinge point 206. Therefore, an end surface 207 of the distal portion 201 and an end surface 208 of the distal portion 202 move from positions separate from each other to positions corresponding with each other. Therefore, part of biological tissues is separated from tissues around it and is harvested by the harvesting parts 203 and 204 to be harvested as a specimen.

In the present procedure, first a tomographic image of the lung by the X-ray is shot and a local anesthesia is performed on the pharynx and larynx of the patient. In use of the flexible tube assembly 100, the distal end of the inner tube 12 is fixed at a place closest to the distal end of the outer tube 11.

Next, the flexible tube assembly 100 is introduced from the mouth of the patient and is moved to the trachea via the pharynx and the larynx. When the flexible tube assembly 100 is moved to one of the main bronchi branching from the trachea toward the left and right lungs, the distal end of the flexible tube assembly 100 is directed toward either one of the left and right main bronchi by rotating the flexible tube assembly 100 in the circumferential direction while performing rotational operation of the rotating dial 31 of the curving operation unit 30 to curve the distal end of the flexible tube assembly 100 toward the one of the left and right main bronchi. Then, the flexible tube assembly 100 is made to penetrate to a further peripheral region. At the position of the main bronchus, the outer tube 11 can be made to penetrate together with the inner tube 12 although depending on the patient.

At a peripheral site on the distal side relative to the superior lobe bronchus, the middle lobe bronchus, or the inferior lobe bronchus on the deeper side of the main bronchus, the sectional area in the conduit is smaller than at the main bronchus and it becomes difficult to make the outer tube 11 having a comparatively-large diameter penetrate in some cases. In this case, the release levers 56 of the switching operation unit 50 are gripped with the opposite hand to the hand holding the curving operation unit 30 and the fixing between the outer tube 11 and the inner tube 12 is released.

This makes it possible for the inner tube 12 to act independently of the outer tube 11. In this state, the slide members 71 and 72 are pushed toward the distal side while the cases 53 and 54 of the switching operation unit 50 are prevented from moving in the longitudinal direction. This causes the inner tube 12 to further protrude from the distal end of the outer tube 11. Then, the penetration direction in which the flexible tube is made to penetrate is checked with an X-ray image or the like and the flexible tube assembly 100 itself is rotated in the circumferential direction while the rotating dial 31 of the curving operation unit 30 is rotated to curve the distal end of the inner tube 12. Then, the distal part of the flexible tube assembly 100 is made to penetrate to a further peripheral site of the superior lobe bronchus or the like.

After the distal end of the inner tube 12 has reached a desired site, the forceps 200 are inserted from the insertion tube 71c and are made to pass through the internal lumen of the inner tube 12 to be pushed forward to the distal part of the inner tube 12. Then, with checking with an X-ray image or the like, the end surface 207 and the end surface 208 of the forceps 200 are brought close to each other from the separated state to clamp lesion tissues. Therefore, a specimen is harvested in the harvesting parts 203 and 204.

After the harvesting of a specimen ends, the flexible tube assembly 100 is removed while the curving direction of the flexible tube assembly 100 and the amount of protrusion of the inner tube 12 from the outer tube 11 are adjusted by the curving operation unit 30 and the switching operation unit 50 so that the occurrence of a pneumothorax and bleeding may be avoided.

The flexible tube such as a catheter used for an endoscope or the like used in the biopsy technique generally has substantially the same diameter from the proximal side to the distal side. However, the size of lumens in the living body into which the medical instrument such as an endoscope is introduced is not uniform in the longitudinal direction. For example, the lung has a structure in which the sectional area decreases along with transition from the central part to the peripheral part like transition from the trachea to the main bronchus, the superior lobe bronchus, the middle lobe bronchus, the inferior lobe bronchus, and so forth.

In the related-art procedure in which an endoscope or the like formed of a flexible tube is used, the flexible tube is selected according to the size of a lumen at the target site. However, in the case of inserting an endoscope into a lung and inserting the flexible tube into a peripheral site such as the superior lobe bronchus and the middle lobe bronchus among organs in the lung, the flexible tube having a comparatively-small diameter is selected. At the central part, the distance between the small-diameter flexible tube and the inner wall of the lung is comparatively long and it is difficult to curve an endoscope or the like having the flexible tube in such a manner as to direct it toward a peripheral site. Therefore, it may be impossible to rapidly move the endoscope having the flexible tube to the desired site.

In contrast, in the flexible tube assembly 100 according to the present embodiment, the tubular member of the flexible tube assembly 100 is composed of double tubes, the outer tube 11 and the inner tube 12, and switching can be made by the switching operation unit 50 between the state in which the outer tube 11, which is not connected to the curving operation unit 30, is fixed to the inner tube 12, which is connected to the curving operation unit 30, and the state in which the fixing is released. Therefore, if the outer tube 11 is made to penetrate together with the inner tube 12 at the central part such as the main bronchus among organs in the lung, the flexible tube assembly 100 can be rapidly directed toward a peripheral site and made to penetrate thereto because the outer tube 11 has a shorter distance from the inner wall of the lung than the inner tube 12. Furthermore, at a peripheral site relative to the superior lobe bronchus or the like, the inner tube 12 can be protruded from the outer tube 11 by the switching operation unit 50 and the slide unit 70 and be made to rapidly penetrate to a target site.

As described above, the flexible tube assembly 100 according to the first embodiment has the double tube 10 having the outer tube 11 and the inner tube 12 and the hand operation unit 20 that is provided on the proximal side of the double tube 10 and operates the outer tube 11 and the inner tube 12. The hand operation unit 20 has the curving operation unit 30 that is connected to the inner tube 12 and curves the inner tube 12, the slide unit 70 that causes slide movement of the inner tube 12 relative to the outer tube 11, and the switching operation unit 50 that makes switching between the state in which the outer tube 11 and the inner tube 12 are fixed to each other and the state in which the fixing is released.

In the case of making the flexible tube assembly 100 enter a lung, by making switching between the fixing between the outer tube 11 and the inner tube 12 and the release of the fixing by the switching operation unit 50 as above, the outer tube 11, whose distance from the inner wall of the lung is comparatively short, can be curved together with the inner tube 12 at the central part so as to be moved forward to a peripheral site. Furthermore, at a further peripheral site relative to the superior lobe bronchus or the like, the fixing between the outer tube 11 and the inner tube 12 is released by the switching operation unit 50 and the inner tube 12 is further protruded from the outer tube 11 toward the distal side by the slide unit 70, so that only the inner tube 12 can be curved by the curving operation unit 30 so as to be made to penetrate to a further peripheral site. Thus, the flexible tube assembly 100 can be made to rapidly penetrate to the desired site.

Moreover, at a site where the lumen is comparatively large, such as the main bronchus, the outer tube 11 is curved together with the inner tube 12. At a further peripheral site relative to the main bronchus, the fixing between the outer tube 11 and the inner tube 12 is released by the switching operation unit 50 to protrude the inner tube 12 and only the inner tube 12 is curved by the curving operation unit 30. Therefore, the axis lines of the distal parts of the outer tube 11 and the inner tube 12 can be oriented in different directions.

Due to the orienting of the axis lines of the distal parts of the outer tube 11 and the inner tube 12 in different directions in this manner, the flexible tube assembly 100 can be accurately curved along the conduit that intricately meanders from the trachea to a peripheral site as in the lung. Thus, a procedure such as harvesting or excision of a lesion site can be performed with a reduced risk of the occurrence of a pneumothorax, bleeding, and so forth due to the insertion of the flexible tube assembly.

Furthermore, the wire 32 of the curving operation unit 30 is connected to the inner tube 12. The switching operation unit 50 has the fixing parts 55 that are connected to the outer tube 11 with the intermediary of the cases 53 and 54 and fix the outer tube 11 to the inner tube 12 by pressing the inner tube 12 radially inward to grip it, the biasing members 52 that are connected to the fixing parts 55 and give the fixing parts 55 a force to fix the outer tube 11 to the inner tube 12, and the release levers 56 that displace the fixing parts 55 radially outward against the force given by the biasing members 52 to release the fixing between the outer tube 11 and the inner tube 12.

Therefore, when the release levers 56 are not gripped, the fixing parts 55 connected to the outer tube 11 with the intermediary of the cases 53 and 54 are biased by the biasing members 52 and grip the inner tube 12. Therefore, the outer tube 11 and the inner tube 12 are fixed to each other. When the release levers 56 are gripped, the force to bias the fixing parts 55 radially inward, given by the biasing members 52, are canceled out and the fixing parts 55 are displaced radially outward, so that the fixing between the outer tube 11 and the inner tube 12 is released.

In this manner, by the simple operation of gripping the release levers 56, the magnitude of the diameter of the distal part of the flexible tube assembly can be easily changed through the fixing between the outer tube 11 and the inner tube 12 and the release of the fixing. Thus, the flexible tube assembly 100 can be made to rapidly penetrate to a target site even in a conduit whose sectional area changes like the lung.

The slide unit 70 has a nesting structure composed of the slide members 71 to 73. Therefore, when the slide members 71 and 72 are brought closer to the cases 53 and 54, the slide members 72 and 73 are housed in the internal space 74 of the slide member 71 and the space occupied by the slide members 71 to 73 can be decreased, which can make the operability favorable.

The slide members 71 to 73 are so configured as to be capable of getting closer to or remoter from each other in the extension direction of the outer tube 11 and the inner tube 12.

Therefore, the inner tube 12 makes slide movement in the same direction as the direction in which the slide members 71 to 73 are caused to get closer to or remoter from each other. Thus, the slide movement can be smoothly carried out without uselessly curving the inner tube 12 in the slide movement.

The curving operation unit 30 is connected to the inner tube 12 in the double tube 10 by the wire 32 and is so configured that the inner tube 12 connected to the curving operation unit 30 curves through pulling operation of the wire 32. Therefore, the curving operation of the inner tube 12 can be rapidly carried out by the simple structure with the pulling operation of the wire 32.

The outer tube 11 is so configured as to have a bellows structure. Therefore, the curving action of the inner tube 12, which is disposed inside the outer tube 11 and is connected to the curving operation unit 30, can be transmitted to the outer tube 11 with high sensitivity. Thus, the outer tube 11, which is not connected to the curving operation unit 30, can be flexibly curved in conformity with a living body.

### Second Embodiment

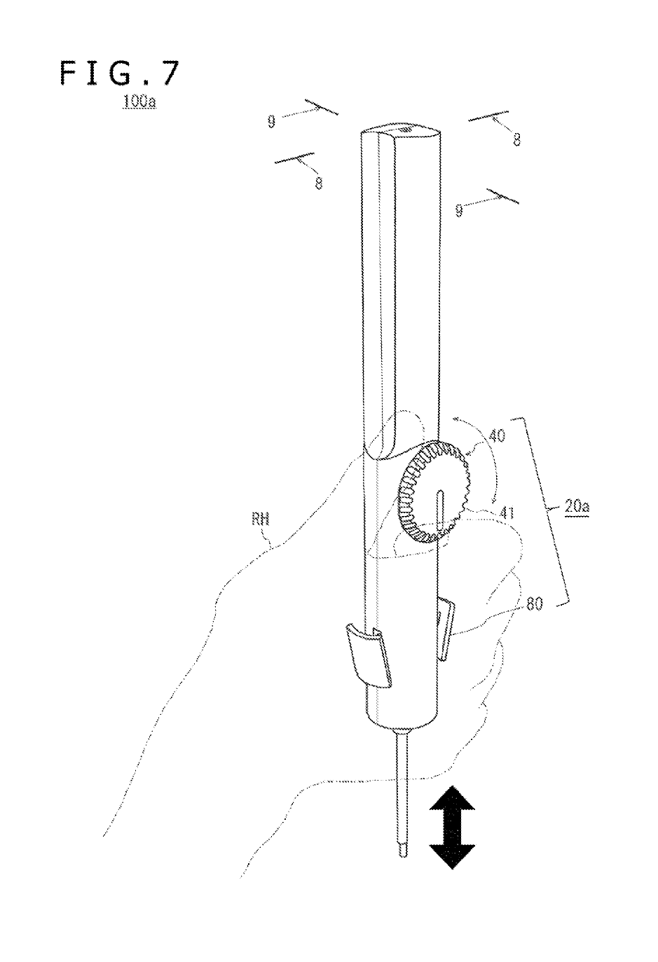

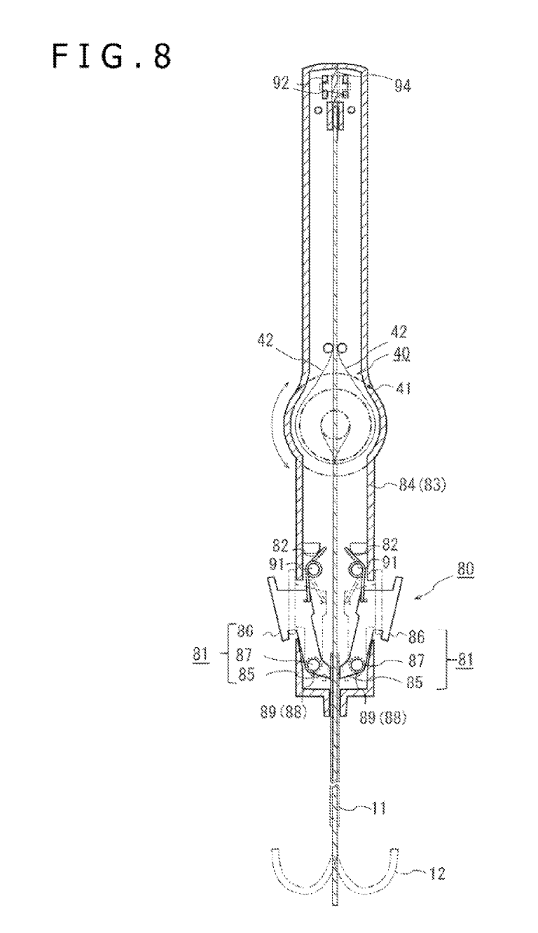

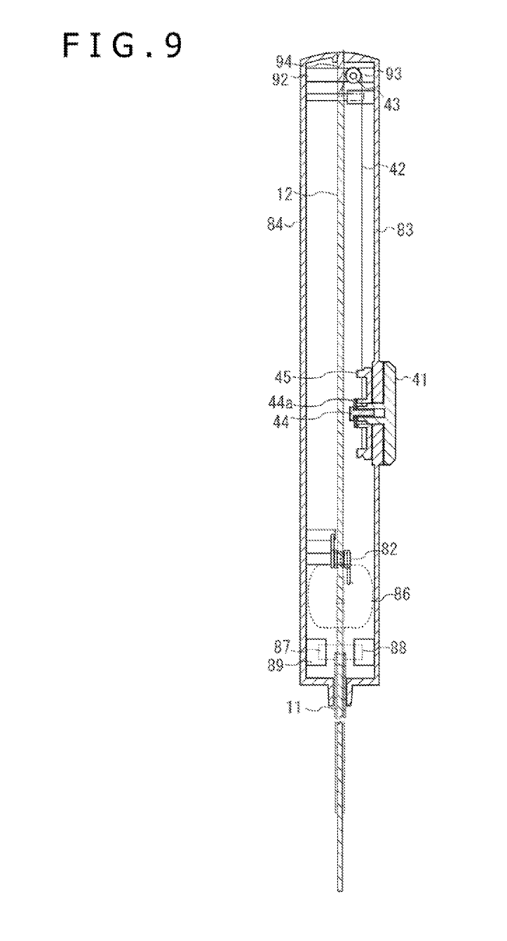

FIG. 7 is a perspective view showing a flexible tube assembly according to a second embodiment. FIG. 8 is a sectional view along line 8-8 in FIG. 7. FIG. 9 is a sectional view along line 9-9 in FIG. 7. In the first embodiment, by causing the slide members 71 to 73 to make slide movement, the inner tube 12 is slide-moved relative to the outer tube 11. However, it is also possible to make the slide movement of the inner tube 12 relative to the outer tube 11 in the following way. Note that the same configuration as that in the first embodiment is given the same symbol and description thereof is omitted.

In the second embodiment, a hand operation unit 20a has a curving operation unit 40 and a switching operation unit 80. The curving operation unit 40 has a rotating dial 41 (equivalent to the rotational operation part) for curving the outer tube 11 and the inner tube 12 and a wire 42 that transmits the motion of the rotating dial 41 to the inner tube 12. The curving operation unit 40 further has a pulley 43 that restricts the position or direction of the extension of the wire 42 and a holding member 45 for rotatably holding the rotating dial 41 onto a case 83 to be described later.

The rotating dial 41 curves the inner tube 12 connected to the wire 42 through rotational operation similarly to the first embodiment. The rotating dial 41 is rotatably attached to the case 83 through the following process. Specifically, a bolt-attached part of the rotating dial 41 is inserted into a through-hole of the case 83 as a constituent element of the switching operation unit 80. In this state, the bolt-attached part of the rotating dial 41 is inserted into a through-hole of the holding member 45. Then, a washer 44a is attached and the rotating dial 41 is fastened by a fastening measure such as a bolt 44. The outer circumference of the rotating dial 41 is formed into a recess-projection shape so that slipping may be suppressed in pressing with a finger.

Part of the wire 42 is bonded to the rotating dial 41 similarly to the first embodiment. A site on the wire 42 remoter from the rotating dial 41 than the pulley 43 is clamped by the first inner tube 12a and the second inner tube 12b of the inner tube 12 and is bonded to the first inner tube 12a by an adhesive or the like. The wire 42 is changed in the extension direction by the pulley 43 and is bonded to the rotating dial 41 with the intermediary of the pulley 43 to thereby be so held as to give a certain tensile force to the inner tube 12.

The pulley 43 is rotatably held by pulley support parts 92 and 93 provided inside cases 83 and 84 as constituent elements of the switching operation unit 80. The pulley 43 is provided at the place on the proximal-most side in the longitudinal direction among places on the route of the wire 42. Therefore, the number of pulleys 43 is one differently from the first embodiment.

To the inner surface of the proximal side of the first inner tube 12a, an insertion tube 94 for inserting a medical instrument such as an endoscope into the inside of the inner tube 12 is connected. The insertion tube 94 is connected by an adhesive or the like on the proximal side of the case 83 in such a manner that the outer tube 11 and the inner tube 12 can be curved by the wire 42. Similarly to the first embodiment, in FIGS. 8 and 9, for convenience of diagrammatic representation, the first inner tube 12a and the second inner tube 12b are shown as a solid monolithic member and the insertion tube 94 is shown as a solid member.

The switching operation unit 80 has grip members 81, biasing members 82, and the cases 83 and 84. The grip members 81 have fixing parts 85, release levers 86, and rotation pins 87 similarly to the first embodiment. The cases 83 and 84 have pin support parts 88 and 89 and holding pins 91 similarly to the first embodiment.

In the second embodiment, the fixing parts 85 do not directly grip the inner tube 12 but press and grip the outer tube 11. In addition, the fixing parts 85 grip the inner tube 12 with the intermediary of the outer tube 11.

When the release levers 86 are not gripped with a hand, the biasing members 82 press the vicinities of the release levers 86 of the grip members 81 radially outward similarly to the first embodiment, and the fixing parts 85 are biased radially inward due to the rotation of the grip members 81 about the rotation pins 87 as pivot points. If the fixing parts 85 are biased radially inward, the fixing parts 85 displace the outer tube 11 radially inward to grip the outer tube 11 and the inner tube 12. Therefore, the outer tube 11 is fixed to the inner tube 12.

Conversely, when the release levers 86 are gripped with a hand, the release levers 86 are displaced radially inward against the radially-outward biasing force to the vicinities of the release levers 86 by the biasing members 82. The fixing parts 85 are displaced radially outward in contrast to the release levers 86 due to the rotation of the grip members 81 about the rotation pins 87 as pivot points. Therefore, the gripping of the outer tube 11 and the inner tube 12 by the fixing parts 85 is released and the outer tube 11 gets separated from the inner tube 12. This releases the fixing between the outer tube 11 and the inner tube 12.

The second embodiment is different from the first embodiment in that the fixing parts 85 do not directly grip the inner tube 12. However, in the second embodiment, the biasing members 82, the release levers 86, the rotation pins 87, the pin support parts 88 and 89, and the holding pins 91 have the same functions as those in the first embodiment and therefore description thereof is omitted.

In the second embodiment, the outer tube 11 is held by the fixing parts 85 so as not to fall off from the cases 83 and 84. When the amount of protrusion of the inner tube 12 from the outer tube 11 is changed, the operator grips the release levers 86 with e.g. a right hand RH as shown in FIG. 7 and sends the outer tube 11 into the inside of the cases 83 and 84 with a left hand LH. Therefore, the amount of protrusion of the inner tube 12 from the distal end of the outer tube 11 increases corresponding to the amount of sending of the outer tube 11 toward the proximal side.

The distal part of the inner tube 12 may be protruded from the distal part of the outer tube 11 by releasing the fixing between the outer tube 11 and the inner tube 12 by the switching operation unit 80 and inserting a flexible tube assembly 100a into a living body. The outer tube 11 has a comparatively-large diameter and thus cannot penetrate to a site whose sectional area is small like the superior lobe bronchus or the like. Therefore, merely through the insertion of the flexible tube assembly 100a, the outer tube 11 becomes incapable of penetrating to the peripheral side where the sectional area is small and only the inner tube 12 can penetrate to the peripheral side. This can change the amount of protrusion of the distal part of the inner tube 12 from the distal part of the outer tube 11.

Furthermore, as shown in FIG. 7, the rotational operation of the rotating dial 41 can be performed by pressing it with e.g. the thumb of the right hand RH and the release levers 86 are so disposed as to be allowed to be gripped by index finger, middle finger, ring finger, and little finger. That is, the rotating dial 41 and the release levers 86 are so configured as to be disposed at such positions that they can be gripped with a single hand.

Therefore, the curving operation by the rotating dial 41 and the advancing/retreating movement operation by the release levers 86 can be carried out without changing the way of holding the flexible tube assembly with the hand. Thus, the operator can concentrate on a procedure such as harvesting or excision of a lesion site without being excessively preoccupied with operation of the flexible tube assembly 100a.

The usage method of the flexible tube assembly 100a according to the second embodiment is similar to that of the first embodiment except for the above-described adjustment of the amount of protrusion of the inner tube 12 from the outer tube 11 and therefore description thereof is omitted.

As described above, in the flexible tube assembly 100a according to the second embodiment, the rotating dial 41 of the curving operation unit 40 is rotatably attached to the case 83 as the grip part in the hand operation unit 20a and the release levers 86 of the grip members 81 forming the switching operation unit 80 are radially displaceably attached to the cases 83 and 84. Furthermore, the rotating dial 41 and the release levers 86 are so configured as to be disposed at such positions that they can be gripped with a single hand. Therefore, with the cases 83 and 84 gripped with a single hand, the curving operation and the advancing/retreating movement action of the inner tube 12 relative to the outer tube 11 can be performed without changing the way of holding the flexible tube assembly with the single hand. This prevents the operator from being excessively preoccupied with the curving operation and the slide movement operation and allows the operator to concentrate on the procedure.

Furthermore, in the flexible tube assembly 100a, slide members like those in the first embodiment are not provided for the slide movement of the inner tube 12 relative to the outer tube 11 and the outer tube 11 itself is moved to advance and retreat relative to the inner tube 12. Therefore, without especially providing a slide movement mechanism in the flexible tube assembly 100a, the slide movement of the inner tube 12 relative to the outer tube 11 can be performed by the simple structure.

The present disclosure is not limited only to the above-described embodiments and various modifications can be made within the scope of claims.

As the first embodiment, an embodiment is described in which the forceps 200 are disposed as a biopsy instrument inside the inner tube 12 of the flexible tube assembly 100. However, the configuration is not limited thereto. An imaging measure such as a camera may be insertably and removably disposed in the inner tube 12 and the outer tube 11 may be provided with a light guide for increasing the amount of light in order to illuminate a target site.

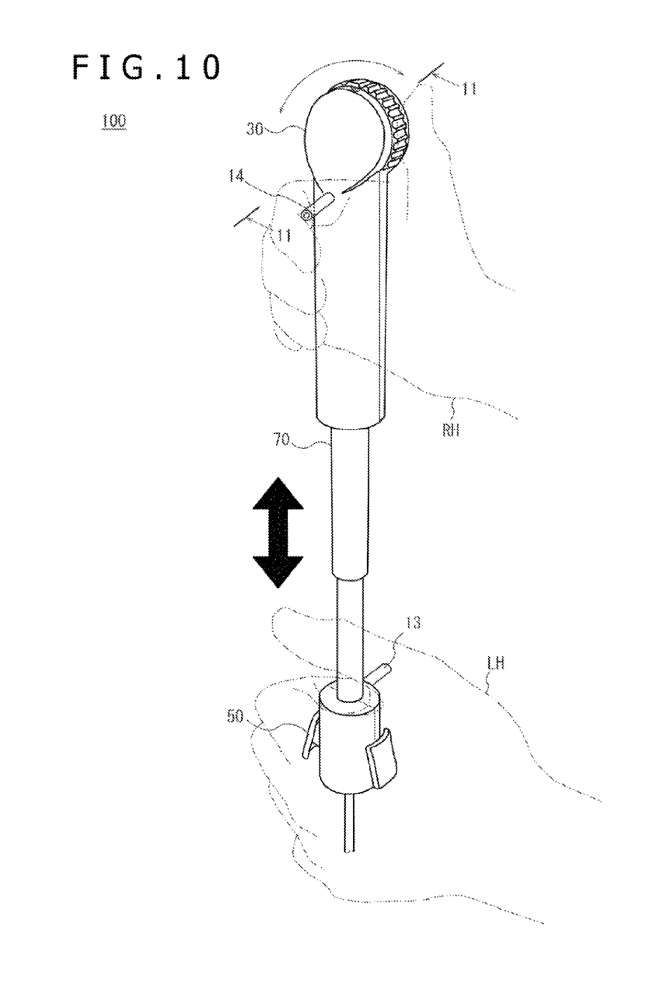

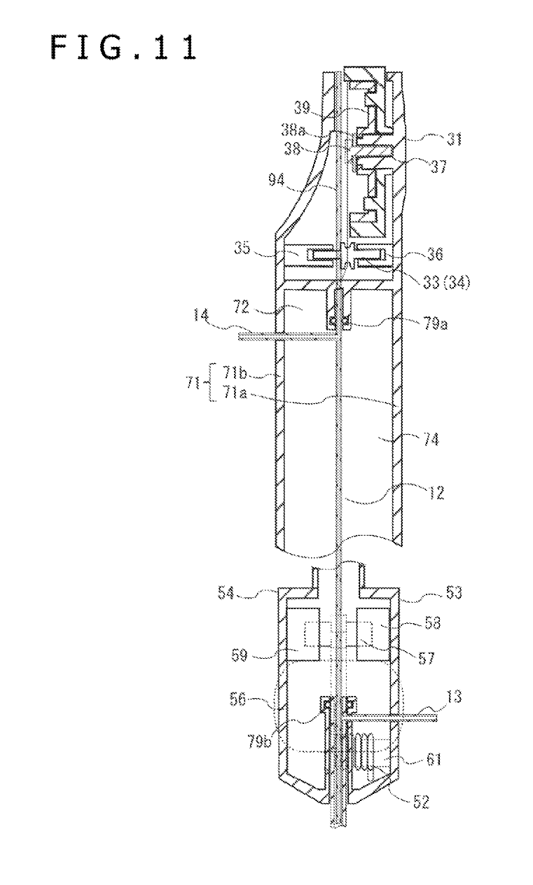

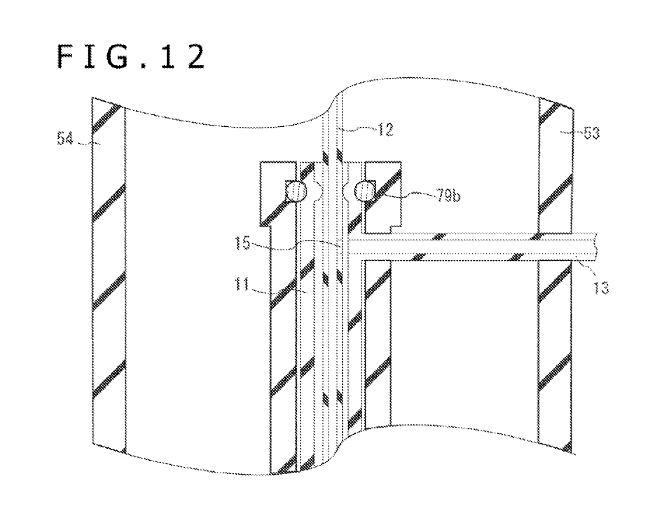

FIG. 10 is a perspective view showing a modification example of the flexible tube assembly according to the first embodiment. FIG. 11 is an enlarged sectional view along line 11-11 in FIG. 10. FIG. 12 is a detail view showing another modification example of the flexible tube assembly and showing the switching operation unit in FIG. 11 in an enlarged manner.

As shown in FIG. 11, a tube 14 is joined to a side surface of the proximal side of the inner tube 12 and protrudes from a side surface of the slide member 71. The internal lumen of the tube 14 communicates with the internal lumen of the inner tube 12.

Furthermore, a tube 13 is joined to a side surface of the proximal side of the outer tube 11 and is so configured as to protrude from the side surface of the case 53. The internal lumen of the tube 13 communicates with the internal lumen of the outer tube 11.

In addition, an O-ring 79a is provided on the proximal side relative to the connected part of the inner tube 12 to the tube 14 and an O-ring 79b is provided on the proximal side relative to the connected part of the outer tube 11 to the tube 13.

Due to the placement of the tube 14 through the slide member 71 in this manner, the tube 14 can be connected to a suction measure that sets the pressure of the internal space to a negative pressure to generate a suction force by a suction pump or the like. Similarly, the placement of the tube 13 through the case 53 allows a similar suction measure to be connected to the tube 13.

This can suck a fluid such as a liquid from the distal side of the inner tube 12 and the outer tube 11.

Although the suction measure is connected to the tube 14, the O-ring 79a is placed on the proximal side relative to the connected part of the inner tube 12 to the tube 14. Therefore, while the sliding of the inner tube 12 is permitted at the part where the O-ring 79a is placed, the sucked fluid does not flow to the proximal side relative to this connected part. The fluid sucked from the distal side of the inner tube 12 flows to the tube 14 from the connected part of the inner tube 12 to the tube 14.

Similarly, although the suction measure is connected to the tube 13, the O-ring 79b is placed on the proximal side relative to the connected part of the outer tube 11 to the tube 13. Therefore, while the sliding of the outer tube 11, the inner tube 12, and a medical instrument inserted into the inner tube 12 is permitted at the part where the O-ring 79b is placed, the fluid sucked from the distal side of the outer tube 11 does not flow to the proximal side relative to this connected part. The fluid sucked from the distal side of the outer tube 11 flows to the tube 13 from the connected part of the outer tube 11 to the tube 13.

Because the flexible tube assembly 100 is introduced into a living body, examples of the fluid sucked by the suction measure include gases existing in the body and liquids such as viscous secretions like sputum.

As above, secretions and so forth in a living body can be sucked by connecting the tube 14 to the inner tube 12 and connecting a suction measure to the tube 14 and connecting the tube 13 to the outer tube 11 and connecting a suction measure to the tube 13.

In the case of attaching imaging apparatus to the flexible tube assembly 100 to make an endoscope, if secretions or the like in a living body exist around a procedure-target site, the secretions or the like often preclude sufficient recognition of the procedure-target site by use of an image.

Even in this case, because the secretions or the like in the living body can be sucked by the suction measure, the image recognition of the procedure-target site can be clearly carried out.

In the above description, grooves for attaching the O-rings are made in the slide member 71 and the cases 53 and 54. However, without making the grooves, the O-rings may be directly bonded to the inner tube 12 and the outer tube 11 by an adhesive or the like.

Furthermore, although the O-rings 79a and 79b are used in order to prevent the fluid sucked by the suction measure from flowing to the proximal side relative to the connected part to the tube, other sealing members may be used as long as the relevant sites can be sealed.

In addition, although the tube to set the pressure of the internal lumen to a negative pressure is connected to both the inner tube 12 and the outer tube 11 in FIGS. 10 and 11, the tube may be connected to only either one.

Moreover, in FIGS. 10 and 11, the tube 14 is connected to the inner tube 12 in order to guide the fluid sucked from the inner tube 12 to the proximal side. However, without connecting the tube 14 to the inner tube 12, an opening 15 may be formed near the connected part of the outer tube 11 to the tube 13 in the longitudinal direction of the inner tube 12 as shown in FIG. 12.

Due to the formation of the opening 15 in the inner tube 12 in this manner, the fluid sucked from the distal side of the inner tube 12 gets out of the opening 15 and passes through the internal lumen of the outer tube 11 to flow to the tube 13. Therefore, secretions in the living body are sucked. Furthermore, the fluid sucked from the distal side of the outer tube 11 flows to the tube 13 from the connected part to the tube 13 as with the above description.

By forming the opening 15 in the inner tube 12 as above, secretions sucked from the distal side of the inner tube 12 can be expelled from the inside of the body through the tube 13 connected to the outer tube 11 without connecting the tube 14 to the inner tube 12.

Although the embodiment in which the suction measure is connected to the flexible tube assembly 100 according to the first embodiment is described, the suction measure may be connected to the flexible tube assembly 100a according to the second embodiment. In the second embodiment, the fixing parts 85 grip both the outer tube 11 and the inner tube 12 and thus a sucked fluid can be prevented from flowing to the proximal side by the fixing parts 85 without providing an O-ring. Even in this case, O-rings may be provided on the proximal side of the inner tube 12 and the outer tube 11 similarly to the flexible tube assembly 100 of the first embodiment.

Furthermore, in the case of connecting the tube 13 to the outer tube 11 and connecting the tube 14 to the inner tube 12, besides connecting a suction measure, water delivery to a procedure-target site may be performed by using a centrifugal pump or the like.

In addition, although the embodiment in which the curving operation unit 30 curves the inner tube 12 is described, a mechanism that curves the outer tube 11 in addition to the inner tube 12 by pulling operation with a wire or the like may be provided.

Moreover, in the first embodiment, the fixing parts 55 of the grip members 51 connected to the outer tube 11 with the intermediary of the cases 53 and 54 fix the outer tube 11 and the inner tube 12 to each other by gripping the inner tube 12.

However, the configuration is not limited thereto. Also in the flexible tube assembly 100 of the first embodiment, the fixing parts 55 may fix the outer tube 11 and the inner tube 12 to each other by gripping the outer tube 11 to grip also the inner tube 12 with the intermediary of the outer tube 11 similarly to the flexible tube assembly 100a of the second embodiment. It should be understood by those skilled in the art that various modifications, combinations, sub-combinations and alterations may occur depending on design requirements and other factors insofar as they are within the scope of the appended claims or the equivalents thereof.

While illustrative and presently preferred embodiments of the present invention have been described in detail herein, it is to be understood that the inventive concepts may be otherwise variously embodied and employed without departing from the spirit and scope of the invention. The appended claims are intended to be construed to include such variations and equivalents.

What is claimed is:

1. A flexible tube assembly comprising:
a multiple tube having at least a first tubular member that has an elongated shape and is hollow and a second tubular member that has an elongated shape and is disposed inside the first tubular member in such a manner as to be capable of advancing and retreating relative to the first tubular member; and
a hand operation unit that is provided on a proximal side of the multiple tube and operates at least the first tubular member and the second tubular member,
wherein the hand operation unit includes
a curving operation unit that is connected to the first tubular member or the second tubular member and curves the first tubular member or the second tubular member that is connected,
a slide unit that causes the second tubular member to make slide movement relative to the first tubular member, and
a switching operation unit that makes switching between a state in which the first tubular member and the second tubular member are fixed to each other and a state in which fixing between the first tubular member and the second tubular member is released.

2. The flexible tube assembly according to claim 1,
wherein a distal part of the first tubular member and the second tubular member includes axis lines of distal parts of the first tubular member and the second tubular member are oriented in different directions by releasing fixing between the first tubular member and the second tubular member by the switching operation unit to protrude the second tubular member from a distal end of the first tubular member and curving the first tubular member or the second tubular member connected to the curving operation unit.

3. The flexible tube assembly according to claim 1,
wherein the curving operation unit is connected to the second tubular member, and
the switching operation unit includes
a fixing part that is connected to the first tubular member and fixes the first tubular member to the second tubular member by pressing the second tubular member radially inward to grip the second tubular member,
a biasing member that is connected to the fixing part and gives the fixing part a force to fix the first tubular member to the second tubular member, and
a release lever that displaces the fixing part radially outward against the force given by the biasing member to release fixing between the first tubular member and the second tubular member.

4. The flexible tube assembly according to claim 1,
wherein the curving operation unit is connected to the second tubular member and has a rotational operation part that curves the second tubular member through rotational operation, and wherein
the switching operation unit includes
a fixing part that is connected to the first tubular member and displaces the first tubular member radially inward to grip the first tubular member in such a manner as to fix the first tubular member to the second tubular member,
a biasing member that is connected to the fixing part and gives the fixing part a force to fix the first tubular member to the second tubular member, and
a release lever that displaces the fixing part radially outward against the force given by the biasing member to release fixing between the first tubular member and the second tubular member, and
the rotational operation part and the release lever are disposed at such positions as to be allowed to be gripped with a single hand.

5. The flexible tube assembly according to claim 1,
wherein the slide unit has a nesting structure composed of at least two slide members.

6. The flexible tube assembly according to claim 5,
wherein the at least two slide members are capable of getting closer to or remoter from each other in direction of an axis line along which the first tubular member and the second tubular member extend.

7. The flexible tube assembly according to claim 1,
wherein the slide unit causes the first tubular member itself to make slide movement relative to the second tubular member.

8. The flexible tube assembly according to claim 1,
wherein the curving operation unit is connected to the first tubular member or the second tubular member by a wire and curves the first tubular member or the second tubular member connected to the curving operation unit by performing pulling operation of the wire.

9. The flexible tube assembly according to claim 1,
wherein the first tubular member has a bellows structure.

10. The flexible tube assembly according to claim 1,
wherein the first tubular member sucks fluid from a distal side of the first tubular member by being connected to a suction measure that generates a suction force.

11. The flexible tube assembly according to claim 1,
wherein the second tubular member is formed into a hollow shape, and
the second tubular member sucks fluid from a distal side of the second tubular member by being connected to a suction measure that generates a suction force.
